(19) United States
(12) Patent Application Publication
(10) Pub. No.: US 2022/0113291 A1
(43) Pub. Date: Apr. 14, 2022

(54) METHOD AND SYSTEM FOR PATHOGEN DETECTION AND REMEDIATION

(71) Applicant: Healthcare Integrated Technologies Inc., Knoxville, TN (US)

(72) Inventors: Kenneth M. GREENWOOD, Davenport, FL (US); Scott Michael BORUFF, Knoxville, TN (US); Jurgen VOLLRATH, Sherwood, OR (US)

(21) Appl. No.: 17/500,904

(22) Filed: Oct. 13, 2021

Related U.S. Application Data
(60) Provisional application No. 63/204,619, filed on Oct. 14, 2020.

(51) Int. Cl. G01N 33/483; G16H 70/60; G16H 50/30; G16H 50/80; A61L 9/20

(52) U.S. Cl. CPC G01N 33/4833; G16H 70/60; G16H 50/30; G16H 50/80; H04W 4/80; A61L 2209/111; A61L 2209/14; A61L 2209/12; A61L 9/20

(57) ABSTRACT

In a pathogen identification and remediation system and method, pathogens are identified by capturing samples in an air stream, identifying the pathogens and neutralizing them, with the option of assessing risk levels and issuing alarms when certain risk levels are exceeded.

### FIELD OF THE INVENTION

The invention relates to the detection and remediation of pathogens such as viruses, bacteria, and fungi.

### BACKGROUND OF THE INVENTION

We are continuously exposed to bacteria, viruses and fungi. To a large extent our bodies' immune systems are able to deal with such environmental onslaughts. However, some pathogens such as the flu virus and, more recently, Covid-19 can have a more impactful effect on a person. Therefore, one way to protect ourselves and to mitigate the effect is to reduce the number of pathogens in our immediate environment.

Another approach is to identify and isolate infected persons early on and also by reducing the proliferation of pathogens.

### SUMMARY OF THE INVENTION

The present invention provides a system for capturing air samples in an environment, e.g., an apartment or house or a room in a house. This may include an air re-circulation system or the use of one or more fans that suck air into a remediation housing or duct. The housing or duct may include detectors for identifying pathogens and means for killing or otherwise neutralizing or disrupting them, e.g., by disrupting the viral shell of a virus. In the case of viruses, the use of ultraviolet light or a pulsed laser may be used to neutralize the virus.

According to the invention, there is provided a system for reducing the threat posed by pathogens, comprising a pathogen detector for detecting pathogens in an air-sample

The system may further include an air-sample capture device, which may comprise a housing or duct.

Further, according to the invention, there is provided a system for pathogen detection and remediation, comprising: a housing defining at least one chamber with an air inlet and an air outlet; at least one biosensor for detecting at least one type of pathogen, and at least one remediation device for neutralizing one or more types of pathogens.

The system may further include a filter mounted to pass air flow through the filter prior to exiting through the outlet, and it may further include a communication means for transmitting data to a processor. The communications means may include one or more Bluetooth, Zigbee, or Z-Wave transceivers connecting the at least one bio sensor to the processor, and may include a communications hub with Bluetooth, Zigbee, or Z-Wave transceiver for receiving data from the at least one biosensor, and having Wifi connectivity for transmitting the data to a server that includes the processor.

The communications means may also include a receiver connected to at least one laser pulse device for receiving frequency adjustment information to adjust the laser pulse frequency.

The detection may include detection of at least one of concentration, type of pathogen, and pathogen characteristics and parameters.

The remediation devices may include one or more of ultra violet (UV) lights, and laser pulse devices.

The system may, further include a processor and memory configured with machine readable code defining an algorithm to control the processor, wherein the algorithm may define logic for comparing pathogen data received from the at least one biosensor with previously stored information.

The previously stored information may be stored in a database or other memory structure connected to the processor.

The algorithm may also define logic for comparing the effectiveness of laser pulse devices to their frequency for a particular type of pathogen. The logic for comparing the effectiveness of laser pulse devices may include comparing different frequencies to effectiveness data to identify the optimum frequency to neutralize a particular pathogen.

The logic may instruct the processor to issue a message to all laser pulse devices associated with a particular pathogen to adjust their frequency to the optimum frequency.

The algorithm may include logic to identify a risk level based on one or more of geographic spread of a particular pathogen, speed of proliferation, density of pathogen, and known risk to humans or other animals posed by the pathogen based on previously captured information, and issuing at least one warning based on the risk level.

Still further, according to the invention, there is provided a method for detecting and remediating pathogens, comprising the steps of: monitoring air samples with biosensors; neutralizing at least one type of the pathogens identified in the air samples, using one or more remediation devices;

assessing the risk posed by the at least one type of the pathogens to define a risk level, and notifying one or more persons based on the risk level posed by the at least one type of pathogen. Risk may be based on concentration of the pathogen, the nature of the pathogen (e.g. airborne as opposed to direct contact transmission), virulence, effectiveness in neutralizing the pathogen, etc.

The method may further include creating heat maps of regions exceeding a predefined risk level and the associated pathogen, or creating contour maps of the various pathogens detected at various risk levels across a geographic area.

### BRIEF DESCRIPTION OF THE DRAWINGS

FIG. 1 is a depiction of one embodiment of a system of the invention;

FIG. 2 shows one embodiment of a detection and remediation unit of the invention;

FIG. 3 shows another embodiment of a detection and remediation unit of the invention,

FIG. 4 shows yet another embodiment of a detection and remediation unit of the invention, and

FIG. 5 is the logic of one implementation of the present invention.

### DETAILED DESCRIPTION OF THE INVENTION

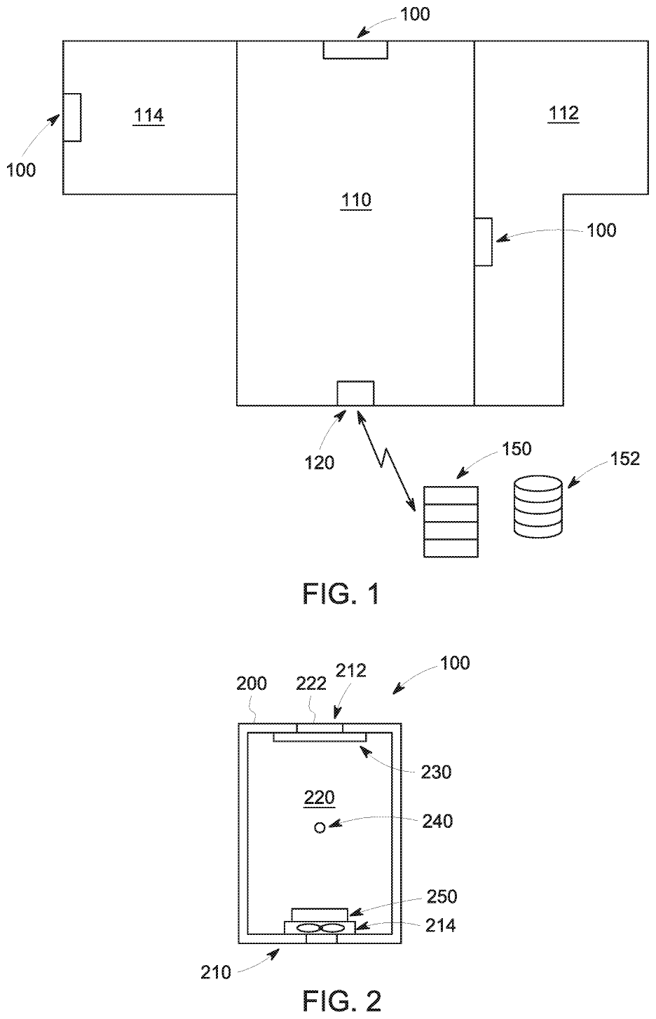

One embodiment of a system of the invention comprises one or more wall-mounted or ceiling-mounted units 100, e.g., mounted in a room 110 of a house, apartment, office etc, as illustrated in FIG. 1.

As shown in FIG. 2, the unit 100 in this embodiment, defines a housing 200 with an air inlet 210 and air outlet 212. The unit includes a fan 214 that sucks air into a chamber 220 defined by the housing 200. The fan 214 creates an air flow from the inlet 210 into the chamber and out through the outlet 212, thereby defining a temporarily-captured air sample within the chamber 220. In order to limit the flow of any pathogens that enter the chamber, back into the room, the outlet 212 includes a filter 222, e.g., a HEPA filter.

The unit 100, further includes a pathogen sensor 230, which may comprise a biosensor and/or optical detector, e.g., the biosensor developed by Swiss Federal Laboratories for Materials Science and Technology, together with ETH Zurich for detecting viral concentration of SARS-CoV-2 (Covid-19 virus) based on the detection of RNA. The coronavirus is a so-called RNA virus: its genome does not consist of a DNA double strand as in living organisms, but of a single RNA strand. The sensor is based. on tiny structures of gold, so-called gold nano-islands, on a glass substrate. Artificially produced DNA receptors that match specific RNA sequences of the SARS-CoV-2 are grafted onto the nano-islands. The receptors on the sensor are therefore the complementary sequences to the virus' unique RNA sequences, which can reliably identify not only concentration but the type of virus. Tests have shown the sensor to be able to distinguish between two very similar RNA sequences of the two viruses.

Where other sensors are use that cannot distinguish between different types of pathogens but are able to extract information about certain characteristics or parameters of the pathogen, this data may be transmitted to a processor for analysis, e.g. by comparing to a database of pathogens and their characteristics and parameters, as is discussed in further detail below.

It will be appreciated, however, that the above optical biosensor is mentioned by way of example only. Other biosensors can be used instead or in addition to the above sensor. These include electrochemical biosensors, which include semiconductors and screen-printed electrodes. These biosensors monitor any alterations in dielectric properties, dimension, shape, and charge distribution as the antibody-antigen complex is formed on the electrode surface. They can be classified into four major groups including potentiometric, amperometric, cyclic voltametric, and impedametric transducers. Another type of biosensor is the piezoelectric biosensor, comprising a quartz crystal microbalance biosensor, which measures any mass change and viscoelasticity of materials by recording frequency and damping change of a quartz crystal resonator. These are, however, highly sensitive to environmental conditions, and require isolation equipment that minimizes vibration. But they have been used effectively in detecting bacteria.

Even though the present invention is described predominantly with respect to viral detection and remediation, it includes the detection and remediation of any pathogens.

In this embodiment the virus sensor 230 is mounted across the outlet 212 to detect viruses flowing from the chamber 220 toward the outlet 212. By mounting the sensor 230 adjacent the filter 222, a greater accumulation of pathogens in and on the filter 222 is available for detection by the sensor 230, and the pathogens are available for a longer period of time.

The present embodiment, further includes a pathogen remediation device 240, which in this embodiment comprises a UV light mounted in the housing. However, other remediation devices may be used that have been found effective in disrupting the viral shell of viruses, such as a pulsed laser. Since the sensor 230 is mounted after pathogens have been exposed to the remediation device 240, the sensor 230 also allows a determination to be made regarding the effectiveness of the remediation device 240 (based on the the relative concentration of live and dead (disrupted) pathogens).

In this embodiment, second virus sensor 250 is also mounted ahead of the remediation device 240 to allow detection and analysis of the pathogen prior to its disruption by the remediation device 240.

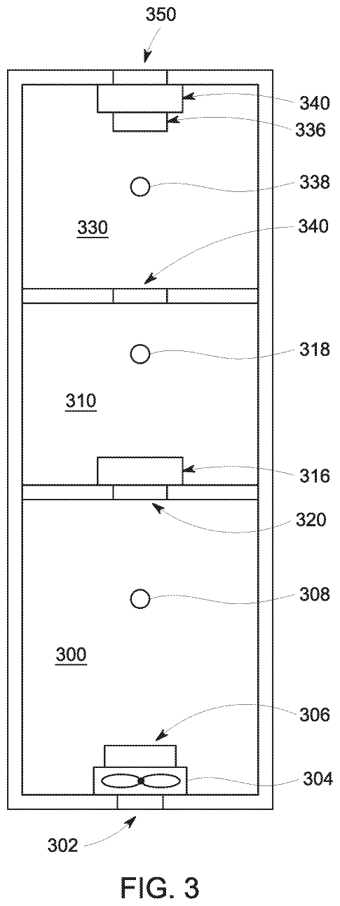

In another embodiment, shown in FIG. 3, the unit 100 comprises a two-chamber housing with a first chamber 300 and a second chamber 310. The first chamber 310 includes an inlet 302 with fan 304 and first pathogen sensor 306. In this embodiment, instead of a UV light, a pulsed laser 308 defines a virus remediation device, as discussed further below. The second chamber 310 is in flow communication with the first chamber 300 by means of the outlet 320 from the first chamber, which also serves as the inlet to the second chamber. The second chamber 310 includes a second pathogen sensor 316 and a second pulsed laser 318.

The effectiveness in disrupting a virus using a pulsed laser involves shaking the virus at its resonant frequency to rupture its capsid (or shell). This resonant frequency will thus depend on the size and mass of the virus, which can be determined by trial and error.

In the present embodiment, however, the trial and error approach is streamlined by collecting data from multiple sensors that monitor the effectiveness of lasers at different frequencies. The determination of the optimum frequency for the remediation of a particular virus can be based on sensors monitoring the effectiveness of pulsed lasers mounted in the same unit or based on lasers mounted in different units.

For example, by analyzing the quantity of viable viruses in each chamber 300, 310, the effectiveness of the first pulsed laser 308 can be determined and the frequency of the second pulsed laser 318, adjusted.

The third chamber 330, which is in flow communication with an outlet 340 from the second chamber 310, includes a third pathogen sensor 336 and pulsed laser 338 for correcting the frequency up or down based on the effectiveness of the adjusted frequency of the second pulsed laser 318. Thus, for example, if the second laser 318 was adjusted up in frequency and failed to reduce the number of pathogens, the third pulsed laser 338 could be adjusted down in frequency or slightly higher in an endeavor to identify the resonant frequency.

Similarly, the effectiveness of remediation devices in other units can be monitored and the optimum frequency associated with a particular virus can be captured in a database and associated with a particular virus. This allows the correct frequency to be dialed in for future cases once a particular virus has been identified, e.g., by an optical sensor.

Any remaining pathogens or at least some of the remaining pathogens are retained in a filter 340 mounted over an outlet opening 350 in the third chamber 330.

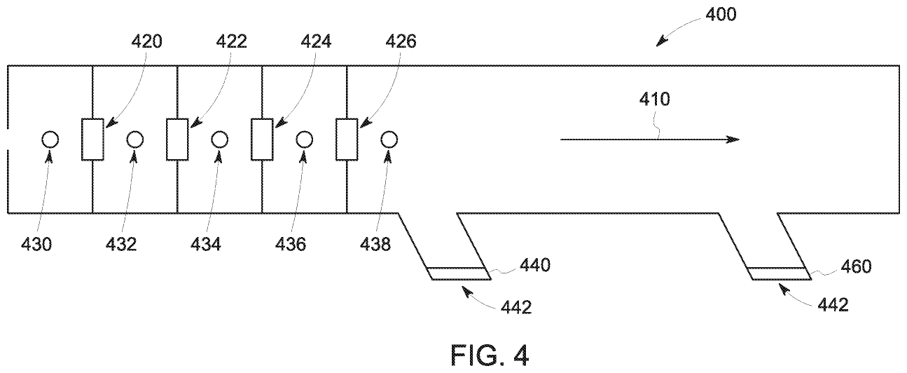

In another embodiment, the unit may be mounted inside or form part of the air-circulation duct work of a building or vehicle. Thus, for example, the air-recirculation ducts in an airplane may include units similar to those described above. Each unit may include a separate housing with one or more chambers, as discussed above, or the duct may define the airflow housing as shown in the embodiment of FIG. 4.

The duct 400 defines an airflow path 410 as depicted by the arrow 410. In this embodiment multiple pathogen sensors 420, 422, 424, 426 are mounted in the duct 400, with interspersed pathogen remediation devices 430, 432, 434, 436, 438 which may comprise pulsed lasers with adjustable frequencies as discussed above, or may comprise different devices for killing or otherwise disrupting different pathogens, e.g., viruses, bacteria, fungi. In this embodiment the duct also includes HEPA filters 440 at the duct outlets 442 for capturing larger particles such as spores or mucous particles entrained with pathogens.

It will be appreciated that the configuration, number, and implementation of the pathogen capture units may vary. Also, the sensors and remediation devices may vary. For example, the sensors may comprise virus sensor based on a biosensor and/or optical detector, e.g., for detecting viral RNA.

As part of the system of the present invention, the sensors include communications means, e.g., Bluetooth, Zigbee, Z-Wave, etc., for communicating data to a hub for processing or for transmitting to a remote location for processing. The system may also include multiple units. For example, in one embodiment, a home implementation may include units placed in various rooms of the home, each of which is in communications with a hub. Referring again to FIG. 1, there is shown an apartment with 3 rooms 110, 112, 114, each with a unit 100, all communicating via Bluetooth with a hub 120. The hub 120 includes a Wifi transceiver for communicating with a remote server 150 with database 152, which may be a dedicated server or comprise a cloud server and database system such as Amazon Web Services (AWS). It will be appreciated that in another embodiment the processing of data can be performed using a local processing unit in the apartment or an edge computing solution or combination thereof.

This allows remote processing of the data received from each sensor e.g., sensor 230 in the FIG. 2 embodiment or sensors 420, 422, 424, 426 in the FIG. 4 embodiment, allowing for the adjustment of laser frequencies as discussed above, and for identifying different pathogens. By identifying the nature of the pathogens, it allows alerts to be generated based on the nature of the threat posed by the pathogen.

In one implementation. the server 150 includes a processor and memory configured with machine readable code to define an algorithm for analyzing the sensor data. The database 152 may include pre-stored data that includes identifying information about various pathogens. Based on a comparison of the data received from the sensors in the various units, to the pre-stored data, the processor makes a determination about the nature, quantity and threat level of any identified pathogen in the housing(s) and generates a corresponding local or more wide-spread alert. For example, an algorithm on the memory may control the processor to generate a local alert by reporting the detection of a virus to a dashboard, and may elevate this to remote alerts, based on a threshold of type of virus and concentration of virus. This combination of type and concentration may vary from pathogen to pathogen, thereby defining the alert threshold setting used by the algorithm in defining the threat level. For example, some viruses are more dangerous and more contagious, and the algorithm controlling the processor would then define a lower required concentration in order to trigger an alarm.

Additionally, if a direct match to an existing pathogen cannot be found in the database, an algorithm (which may comprise an artificial intelligence (AI) system) can attempt to extrapolate from the characteristics of the virus (e.g., its RNA sequence), in order to detect new (unknown) viruses, or mutations of existing viruses, and thereby attempt to positively ID the virus or classify it as a new virus. An algorithm may also identify a pathogen remediation device that is most likely to be effective, as well as its setting, based on data collected for similar pathogens. For instance, a pulsed laser's pulse frequency may be adjusted to a frequency that has been found to be effective for disrupting viruses with a similar RNA sequence.

Also, data relating to the degree to which the pathogen is detected in neighboring units, e.g., in multiple rooms in the same house, or in multiple houses in a neighborhood may be used to create heat maps in order to assess the spread of the pathogen (both the geographic spread and the speed with which it spreads) in order to define the threat of the pathogen, such as the virulence of a virus.

As discussed above, the system may attempt to neutralize the pathogen, e.g., using UV or, in the case of viruses through pulsed laser excitation. The latter has been shown to excite the viral shell through vibration causing it to be compromised, thereby disrupting and neutralizing the virus.

An initial set of frequencies adopted in order to neutralizing the virus can be predicted based on the measured characteristics of the virus, the viral class, and kill-frequency of similar viruses. Thus, the effectiveness of the system can be increased by first analyzing the virus in order to identify the best response.

In one embodiment, the system further seeks to improve the remediation process, by changing the frequencies slightly (performing a frequency scan) and monitoring the effect. Once an optimum frequency has been determined, the processor reports this to the network to reconfigure the other remediation devices dealing with the same pathogen.

It will be appreciated that if the same virus is detected by multiple units (across multiple rooms or residences) simultaneously, the work of testing various frequencies may be distributed across all sensors in the region to detect that virus and work in parallel, each testing a different frequency, in order to more quickly find the neutralizing frequency.

In one embodiment the system includes secondary remediation units and larger ducts for rapidly recirculating and cleaning the air in a room or set of rooms. In another embodiment, once the optimum frequency for a laser pulse system has been determined, a wide-beam laser can be used to radiate an entire room killing the virus throughout the area.

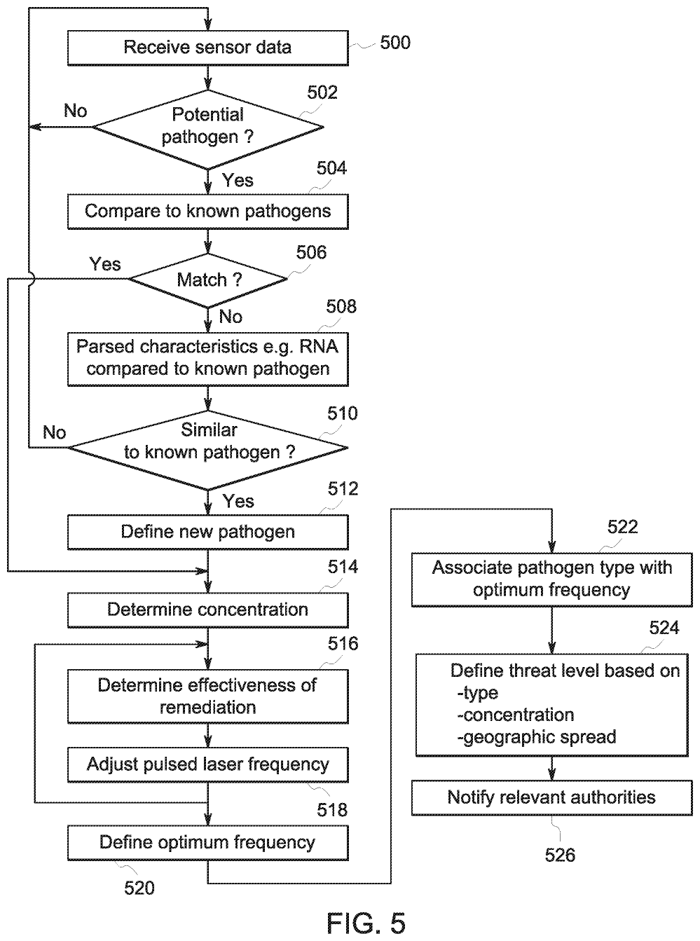

FIG. 5 shows one embodiment of the logic in an algorithm implementing the system of the present invention. In step 500, data is received from a pathogen sensor (also referred to herein as a detector). This data is analyzed for DNA and RNA characteristic of a viral, bacterial or fungal pathogen (step 502). If a potential pathogen is detected in the air sample analyzed by the senor, it is compared to a database of known pathogens (step 504). If there is match the logic jumps to step 514 (discussed below). If no match is found, the parsed characteristics, such as RNA, is analyzed to determine whether it is similar to the RNA sequence or other characteristic of a known pathogen (step 508). In step 510 the similarity determination to a known pathogen is made. If not the logic loops back to analyze the next sample. If there is a similarity, a new pathogen is defined (step 512). The air sample of known pathogens and newly defined pathogens is then analyzed to define the concentration of pathogens in the sample (step 514). The percentage or number of dead or compromised pathogens is then determined in step 516 and, in the case of a pulsed laser remediation device, the laser frequency is adjusted (step 518) to determine whether there is an improvement in the kill rate. This is repeated until an optimum frequency is identified (step 520). The database is then updated by associating pathogen type with optimum laser frequency (step 522). In this embodiment the threat level posed by the pathogen is then determined based on type of pathogen, how wide-spread it is (based on sensor data received from other locations), and in this embodiment, based also on the concentration of the pathogen (step 524). Based on the threat level the appropriate authorized persons or bodies are then notified with details of the threat (step 526).

One aspect of the present invention, thus includes early identification and intervention to avoid epidemics and undue spread of pathogens, By collecting data from sensors distributed over geographic region and identifying the risks posed by identified pathogens, heat maps of high-risk areas can be defined or contour maps created of different pathogens and of different risk levels over the geographic region.

While the present invention was described with respect to specific embodiments, it will be appreciated that the system of the invention can be implemented using different sensors, remediation devices, housing configurations and analysis algorithms and networks without departing from the scope of the invention.

What is claimed is:

1. A system for pathogen detection and remediation, comprising
a housing defining at least one chamber with an air inlet and an air outlet;
at least one biosensor for detecting at least one type of pathogen, and
at least one remediation device for neutralizing one or more types of pathogens.

2. A system of claim 1, further including a filter mounted to pass air flow through the filter prior to exiting through the outlet.

3. A system of claim 1, wherein the detection includes at least one of concentration, type of pathogen, and pathogen characteristics and parameters.

4. A system of claim 1, wherein the remediation devices includes one or more of UV light, and laser pulse device.

5. A system of claim 4, further comprising a communications means for transmitting data to a processor.

6. A system of claim 5, wherein the communications means includes one or more Bluetooth, Zigbee, or Z-Wave transceivers connecting the at least one biosensor to the processor.

7. A system of claim 6, wherein the communications means includes a communications hub with Bluetooth, Zigbee, or Z-Wave transceiver for receiving data from the at least one biosensor, and having Wifi connectivity for transmitting the data to a server that includes the processor.

8. A system of claim 5, wherein the communications means also includes a receiver connected to at least one laser pulse device for receiving frequency adjustment information to adjust the laser pulse frequency.

9. A system of claim 1, further comprising a processor and memory configured with machine readable code defining an algorithm to control the processor.

10. A system of claim 9, wherein the algorithm defines logic for comparing pathogen data received from the at least one biosensor with previously stored information.

11. A system of claim 10, wherein the previously stored information is stored in a database or other memory structure connected to the processor.

12. A system of claim 9, wherein the algorithm defines logic for comparing the effectiveness of laser pulse devices to their frequency for a particular type of pathogen.

13. A system of claim 12, wherein the logic for comparing the effectiveness of laser pulse devices includes comparing different frequencies to effectiveness data to identify the optimum frequency to neutralize a particular pathogen.

14. A system of claim 13, wherein the logic instructs the processor to issue a message to all laser pulse devices associated with a particular pathogen to adjust their frequency to the optimum frequency.

15. A system of claim 9, wherein the algorithm includes logic to identify a risk level based on one or more of: geographic spread of a particular pathogen, speed of proliferation, density of pathogen, and known risk to humans or other animals posed by the pathogen based on previously captured information, and issuing at least one warning based on the risk level.

16. A method for detecting and remediating pathogens, comprises the steps of:
monitoring air samples with biosensors for the presence of one or more pathogens,
neutralizing at least one type of said pathogens identified in the air samples, using one or more remediation devices,
assessing the risk posed by at least said one type of the pathogen to define a risk level, and
notifying one or more persons based on the risk level posed by said at least one type of pathogen.

17. A method of claim 16, further comprising creating heat maps of regions exceeding a predefined risk level and the associated pathogen, or creating contour maps of the various pathogens detected at various risk levels across a geographic area.
